(19) United States
(12) Patent Application Publication
(10) Pub. No.: US 2020/0188288 A1
(43) Pub. Date: Jun. 18, 2020

(54) FINGERNAIL GROWTH AND PROTECTION SYSTEM

(71) Applicant: Marlyn Knight, Clifton, NJ (US)

(72) Inventor: Marlyn Knight, Clifton, NJ (US)

(21) Appl. No.: 16/718,122

(22) Filed: Dec. 17, 2019

Related U.S. Application Data
(60) Provisional application No. 62/780,788, filed on Dec. 17, 2018.

(51) Int. Cl. A61K 8/92; A61K 8/67; A61K 8/65; A61K 8/27; A61K 8/23; A61K 8/44; A61K 8/9789; A61Q 3/02

(52) U.S. Cl. CPC A61K 8/922; A61K 8/678; A61K 8/676; A61K 8/65; A61Q 3/02; A61K 8/23; A61K 8/44; A61K 8/9789; A61K 8/27

(57) ABSTRACT

The invention is directed to a fingernail growth and protection system. The system provides a five-step process using all natural materials to grow and strengthen the nails for use with nail polish and other cosmetic treatments. The process supports the nail with a small paper wedge, trimmed to fit under the nail. The process further provides steps to grow and strengthen the user's nails with soaking, scrubbing, polishing, and oiling steps to produce longer and stronger nails.

### BACKGROUND

### Field of the Invention

The present invention relates generally to cosmetic products and accessories.

More specifically, the present invention is a fingernail growth and protection system.

### Summary of Related Art

Long, elegant nails are an important part of the appearance of millions of women. Unfortunately, such activities as household chores and typing, as well as certain health care issues, present a very serious obstacle to the growth and strength of the nails. Currently, there are numerous products on the market that address the single issue of nail strengthening.

### SUMMARY OF THE INVENTION

Recovery and protection are essential to grow a new nail, in addition to strengthening of the nail. After a nail is broken, protection is essential to guard against further breakage and to encourage new growth. A truly effective nail growth system will promote growth and strengthening of nails, providing a more reliable platform for nail polish and other decorative treatments.

Included herein, is a fingernail growth and protection system, which provides a five-step process using all natural materials to grow and strengthen the nails. According to some embodiments, fingernail growth and protection system is disclosed. The system includes a combination of five treatments, desirably using all natural materials to grow and strengthen the nails and for use with nail polish and other cosmetic treatments. One treatment includes supporting the nail with a substrate such as a small paper wedge, trimmed to fit under the nail. Additional treatments include scrubbing, soaking, polishing, shielding/protection, and oil treatment. The combination of treatments produce longer and stronger nails, compared to the absence of treatment.

Additional features and advantages of the invention will be set forth in the description which follows, and will be apparent from the description, or may be learned by practice of the invention. The foregoing general description and the following detailed description are exemplary and explanatory and are intended to provide further explanation of the invention. Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 provides a front view of the nail oil component of the first exemplary embodiment, displaying the nail oil bottle 12, and the bottle cap 15.

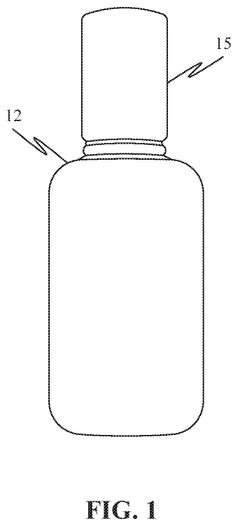

FIG. 2 provides a top perspective view of the nail oil component of the first exemplary embodiment, displaying the nail oil bottle 12, and the bottle cap 15.

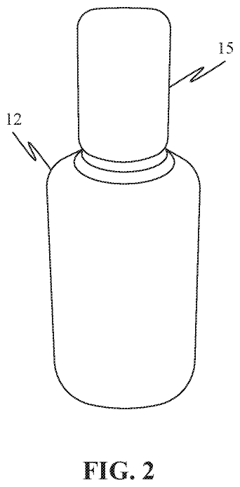

FIG. 3 provides a front view of the nail polish component of the first exemplary embodiment, displaying the nail polish bottle 13, and the bottle cap 15.

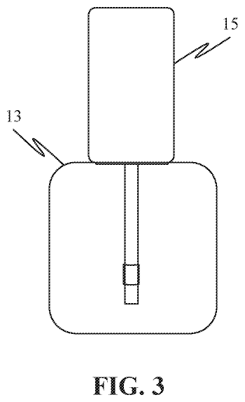

FIG. 4 provides a top perspective view of the nail polish component of the first exemplary embodiment, displaying the nail polish bottle 13, and the bottle cap 15.

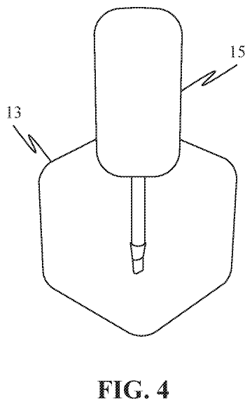

FIG. 5 provides a top perspective view and a front view of the nail scrub component of the first exemplary embodiment, displaying the nail scrub jar 10, and the jar lid 14.

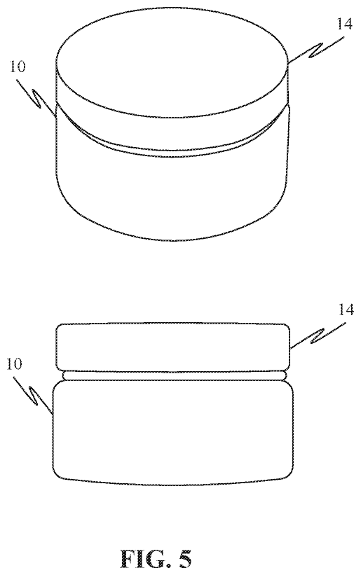

FIG. 6 provides a top perspective view and a front view of the nail soak component of the first exemplary embodiment, displaying the nail soak jar 11, and the jar lid 14.

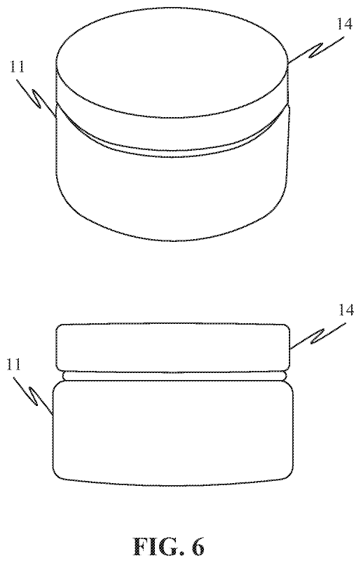

FIG. 7 provides a top view and a side perspective view of the nail support component of the first exemplary embodiment, displaying the nail support 16.

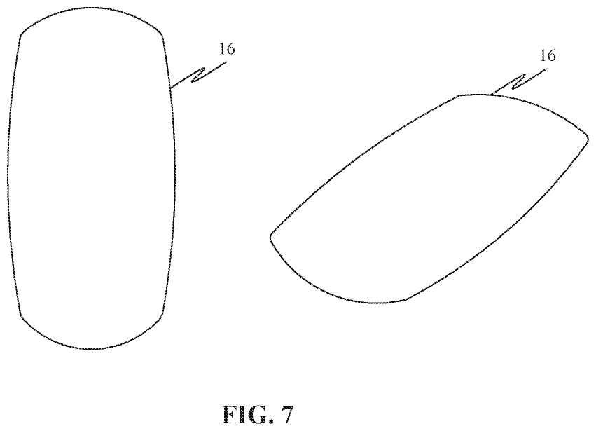

FIG. 8-10 are photos of nails at days 1, 10, and 30 into treatment with the system of one embodiment. This shows that the treatment provided support for nail growth.

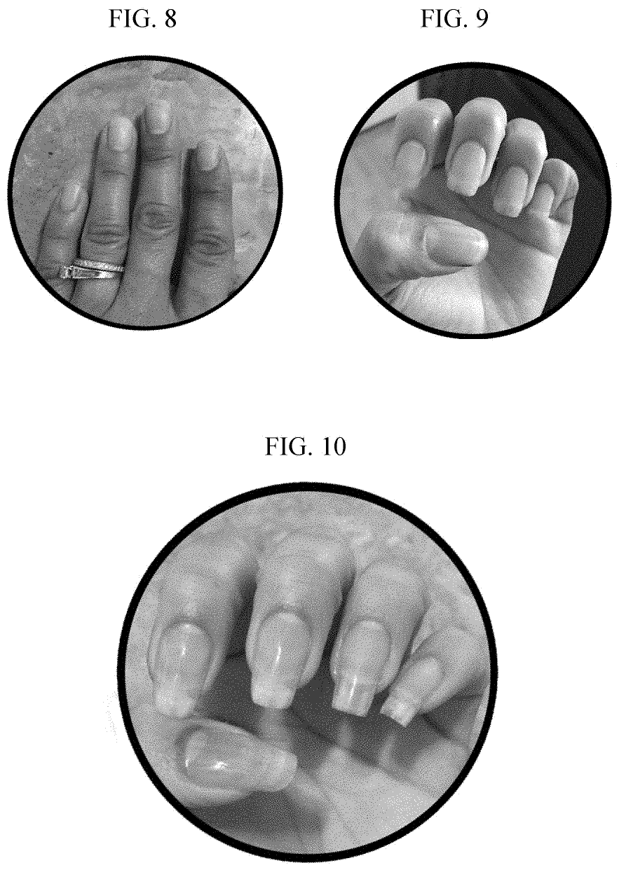

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure.

Additionally, it is important to note that each term used herein refers to that which an ordinary artisan would understand such term to mean based on the contextual use of such term herein. To the extent that the meaning of a term used herein-as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods.

Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of a fingernail growth and protection system, embodiments of the present disclosure are not limited to use only in this context.

Referring now to the invention in more detail, the invention is a fingernail growth and protection system.

One embodiment is includes a system which provides five treatments, corresponding to the following process: nail soak, nail scrub, nail protectors 16, nail polish, and nail oil. The nail protectors 16 are may be manufactured from any suitable material such as a fibrous material. This could be a nonwoven fibrous material similar to that used for tea bags or filters, such as filter paper. The material may be derived from a cellulosic, cotton, silk, hemp, or other organic source. Other suitable materials include porous polymeric material such as polyester and/or nylon. The polymeric materials may be formed into fiber or into a nonwoven sheet. Alternatively, a perforated polymeric sheet may be used. Combinations of one or more fiber or sheet may be included.

A kit of some embodiments may include a plurality of nail protectors 16 of different sized to accommodate different size nails. package containing a large plurality of nail protectors 16 which is a multiple of 10, such as 40 nail protectors 16. Alternatively, the system may provide instructions to the user to make nail protectors. The instruction may include obtaining the appropriate fibrous/perforated material and cutting to the appropriate nail size.

The nail protector 16 may be folded under the nail, with a portion of the nail protector 16 folded over to protect the top surface of the nail. The nail protector 16 may alternately be folded under the nail with the excess material trimmed off. The nail protector provides a shield and/or provide additional support for the nail. An additional alternative includes separate nail protectors applied to each of the top and under surfaces of the nail. Prior to application of the nail protector, the nail polish treatment, described below, is applied to the nail. The nail protector is then applied and smoothed and allowed to dry on the nail.

One or more of the nail treatments selected from the nail soak, nail scrub, or nail oil may alternatively be applied to the inside of a glove. The glove is then worn by the user to transfer the treatment to the nails.

The nail soak component, the nail scrub component, the nail polish component, and the nail oil component may be manufactured from any suitable materials, such as a protein-based precursor comprised of hydrolyzed collagen, methylparaben, propylparaben, and water.

The nail scrub component may be provided in a cylindrical nail scrub jar 10 with a jar lid 14. The user is instructed to apply the nail scrub component to the nails every night, without rinsing. The nail scrub can be massaged into the nails and cuticles until absorbed.

The nail scrub component may be manufactured with the protein-based precursor, castor oil jelly (derived from castor oil and wax), petroleum jelly. The jelly is the primary component present in an amount of greater than 50% by weight of the nail scrub composition, or greater than 80%, or greater than 90% by weight of the nail scrub. Additives may include salt crystals (such as pink salt for exfoliation), zinc powder to support new nail growth and strength, calcium (such as calcium carbonate or calcium citrate) to smooth nails and reduce brittleness, and L-Lysine powder, an essential amino acid, to support collagen growth and nail strength.

The nail soak component may be provided in a cylindrical nail soak jar 11 with a jar lid 14. The nail soak component may be manufactured with the protein-based precursor, castor oil, olive oil, and combinations thereof. The precursor or oil may be present in an amount greater than 50% by weight of the nail soak component, such as greater than 80%, or greater than 90% by weight of the nail soak component. The precursor or oil, or combination there if is combined with one or more additives selected from zinc, calcium, L-Lysine, as described above, optionally with the addition of Vitamin C. About 0.5 teaspoon to about 3.0 teaspoons of the nail soak component are combined with 1 to 4 fluid ounces of warm water. Alternatively 0.5 teaspoon of zinc, calcium, L-Lysine, lime powder and olive oil. The lime powder may be replaced by half of a fresh lime, or other source for Vitamin C. The user is instructed to soak the nails in the nail soak every day for 10 days for a suitable period of time such as about 10 min to about 15 min. For faster results, the user is instructed to soak the nails once weekly after the initial 10-day period is completed.

The nail polish component is provided in a square nail polish bottle 13 with a cylindrical bottle cap 15. The nail polish component may be manufactured with the protein-based precursor, top or base coat nail polish blended with a mixture of 50 percent lime powder and 50 percent unflavored gelatin. Alternatively, the nail polish top or base coat may be combined with vitamin C, vitamin E, and hydrolyzed keratin. Nail polish top and base coat compostions are well-known in the art. One or more top or base coat can be included in combination. Examples include, but are not limited to, aqueous emulsions of acrylic polymer and vinyl acetate/acrylic copolymer, as shown in U.S. Pat. No. 6,391,964B1, latex based compositions, as shown in U.S. Pat. No. 9,636,293B2, and water-soluble or water-swellable polymer dissolved in a solvent, as shown in U.S. Pat. No. 3,928,113A, all of which are incorporated by reference in their entirety.

The nail oil component may be provided in a cylindrical nail oil bottle 12 with a cylindrical bottle cap 15. The nail oil component may be manufactured with the protein-based precursor, olive oil, jojoba oil, castor oil, rosemary oil, lavender oil, lemon oil, calcium, L-Lysine, selenium, unflavored gelatin, whole cloves, zinc, and horsetail powder. Alternatively, the nail oil may include, olive oil, jojoba, castor oil, clove, lemon, with additives optionally including vitamin E, horsetail, selenium, zinc, calcium, and L-Lysine. The user may rub the nail oil component around and under the nails twice daily, or every day, as needed.

The nail soak jar 11, the nail scrub jar 10, the nail polish bottle 13, and the nail oil bottle 12, are preferably manufactured from a rigid, durable material which is transparent, such as glass or plastic. The bottle cap 15 and the jar cap 14 are preferably manufactured from a rigid, durable material such as plastic or aluminum alloy.

Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

1. A kit for promoting nail growth including the following treatments, a nail scrub, a nail soak, application of nail protectors, nail polish treatment, and nail oil.

2. The kit of claim 1, wherein said nail scrub includes castor oil jelly, zinc, calcium, and L-lysine.

3. The kit of claim 1, wherein the nail soak includes olive oil, zinc, calcium, and vitamin C.

4. The kit of claim 1, wherein said nail polish treatment includes a nail polish top or base coat, vitamin C, vitamin E, and hydrolyzed keratin.

5. The kit of claim 1, wherein the nail protector comprises a material selected from tea bag or filter paper.

6. The kit of claim 1, wherein the nail oil comprises olive oil, jojoba, castor oil, clove, lemon, vitamin E, horsetail powder, selenium, zinc, calcium, and L-lysine.

7. A method for promoting nail growth comprising the following treatments, application of a nail scrub, application of a nail soak, application of nail protectors, application of a nail polish treatment, and application of nail oil.

8. The method of claim 7, wherein the application of nail scrub is daily to nails without rinsing.

9. The method of claim 7, wherein the application of nail soak is daily for 10 days.

10. The method of claim 7, wherein the application of nail polish is daily or as needed.

11. The method of claim 7, wherein the application of nail oil is daily or as needed.
